(19) United States
(12) Patent Application Publication
(10) Pub. No.: US 2006/0073610 A1
(43) Pub. Date: Apr. 6, 2006

(54) Patterned composite membrane and stenciling method for the manufacture thereof

(75) Inventor: William Kopaciewicz, West Newbury, MA (US)

Correspondence Address: Legal Division;Millipore Corporation, 80 Ashby Road, Bedford, MA, 01730, US

(73) Assignee: Millpore Corporation

(21) Appl. No.: 10/191,364

(22) Filed: Jul. 8, 2002

Related U.S. Application Data
(63) Continuation of application No. 60/303,678, filed on Jul. 6, 2001.

(51) Int. Cl. G01N 33/543

(52) U.S. Cl. 436/518

(57) ABSTRACT

A patterned composite membrane useful, for example, in proteomic and genomic biopolymer characterization is disclosed. The patterned composite membrane, in general, comprises a substantially planar support and porous material arranged thereon to define a plurality of discrete binding sites. Each binding site is configured such that it will preferentially bind a predetermined proteomic or genomic biopolymeric species (or other object) upon treatment of the patterned composite membrane with a sample solution containing said biopolymeric species (or said other object). A method for the manufacture of a patterned composite membrane is also disclosed. The method, which employs the use of a mask in the formation of a membrane pattern, is particularly well-suited to industrial application involving comparatively large product volume demands.

### REFERENCE TO RELATED APPLICATION

This application claims the benefit of Provisional U.S. Patent Application Ser. No. 60/303,678, filed Jul. 6, 2001.

### FIELD

This invention relates in general to membrane technology, and more particularly, to a patterned composite membrane useful in the detection and/or identification of a predetermined proteomic and genomic biopolymer, or species thereof, or other fluid-borne object.

### BACKGROUND

Research—for example, in the life sciences, biopharmaceutical, semiconductor, and water purification industries—continues to employ and fuel interest in quick, efficient, and inexpensive means for withdrawing particles, biopolymers, microorganisms, solutes, and like objects from liquid and gas fluid streams for the purposes of identification, detection, quantification, and/or like analytical objectives. Myriad analytical tools and protocols capable of providing such functionality are described in the scientific literature. However, precipitated particularly by an escalating interest in so-called proteomic and genomic “microarray” technology, the investigation of the means for and applications of the simultaneous conduct of varied analytical assays on a single unitary medium is noticeably expanding and intensifying.

A typical method for creating a proteomic or genomic microarray is to deposit minute aliquots of differentially-reactive biochemical probe solutions onto a glass slide. The biochemical probes become attached or otherwise fixed to the glass slide, for example, by adsorption or by covalent bonding. In use, the microarray-bearing slide is immersed in, blotted or smeared with, or otherwise exposed to a sample solution. If the sample solution contains the targeted components, those component are selectively withdrawn and captured by the probe (or probes), and thereby, localized for subsequent analysis.

While conventional microarray technology in its current embodiments is and will likely continue to be used to acquire useful analytical information concerning the biochemical constituency of fluid streams, those skilled in the art understand that its use is often constrained (or otherwise effected) by certain factors.

First, it is commonly known that the diffusional spread of a typical biochemical probe solution upon application onto a glass side is often difficult to control. Without good spot control, a resultant microarray can produce unreliable, errant, inaccurate, or otherwise imprecise analytical information.

Second, when a typical biochemical probe solution is applied onto a glass slide, the drop spreads and dries into a thin film on the slide's surface. To avoid overspreading, comparatively minute aliquot volume are customarily used. The results in a thin spot having a comparatively small surface area for sample interaction and a comparatively low concentration of the biochemical probe. The typical processing and reaction time subsequent the exposure of a conventional slide-borne microarray to a sample solution is comparatively long.

Third, the preparation of conventional slide-based microarrays is generally complicated, and hence, is often confined by manufacturers and users to applications calling for very large, dense arrays of biochemical probes (i.e., high information applications). Accordingly, most commercially available microarrays are comparatively expensive and may not be well-suited—in respect of their associated cost and/or functionality—for analytical applications with narrower, more selective detection and/or identification parameters.

Fourth, slide-based microarray technology is generally not versatile; applications thereof being predominantly confined to biochemical analyses.

In light of the above, need exist for a new platform for the conduct of microarray-type analysis for proteomic, genomic, or other applications, the platform being versatile, comparatively inexpensive, easy to manufacture, reasonably accurate, and reasonably sensitive.

### SUMMARY

In light of the above-mentioned need, the present invention provides a patterned composite membrane useful, for example, in proteomic and genomic characterization protocols. The patterned composite membrane 10, in general, comprises a substantially planar support 12 onto which is provided discrete depositions of porous material 14. The discrete depositions 14 can be engineered in respect of its arrangement and/or composition to correspond with the particular chemical and/or mechanical properties of one's desired analytical target(s). Having good design flexibility and potential for user-customization, the present invention encompasses several possible embodiments.

In one preferred embodiment of the present invention, the porous material 14—comprising a plurality of sorptive particles dispersed in a polymeric binder—is arranged on the substantially planar support 12 to define a plurality of discrete binding sites 14. Although the discrete binding sites 14 can have similar or different composition, each is specifically configured to preferentially bind a predetermined biopolymer. Typical target biopolymeric species include proteomic species, such as enzymes, antibodies, peptide hormones, and other like polypeptides; and genomic species, such as oligonucleotides, RNA and DNA, plasmids and plastids, episomes, and other like nucleic acids.

The present invention also provides a method for the manufacture of a patterned composite membrane. The method comprises, in no particular order, the steps of: (a) providing a substantially planar support; (b) providing a membrane precursor solution capable of being processed to form a porous membrane material; (c) overlaying a mask onto said substantially planar support, said mask comprising a substantially flat material with at least one visually-perceptible opening therethrough; (d) depositing said membrane precursor solution onto said substantially planar support through said opening of said overlaying mask; (e) removing said mask from said substantially planar support so that the deposition remains on the support, said deposition corresponding substantially to the shape of said opening of said mask; and (f) processing said membrane precursor solution to form said porous material.

In light of the above, it is a principal object of the present invention to provide a patterned composite membrane comprising porous membrane material deposited discretely on a substantially planar support.

It is another object of the present invention to provide a patterned composite membrane having a predefined arrangement of binding sites, each binding site capable of preferentially withdrawing a predetermined proteomic or genomic biopolymeric species from a biochemical sample solution.

It is another object of the present invention to provide a patterned composite membrane which, when brought into contact with a biochemical sample solution, can yield visually-detectable information regarding the biopolymeric constituency of said solution, as a result of its reaction thereto and subsequent treatment under known post-sampling image development regimens.

It is another object of the present invention to provide a patterned composite membrane comprising a substantially planar support onto which is provided discrete non-contiguous deposits of porous material, each discrete deposit of porous material having a porosity and microstructure capable of selectively admitting and holding a predetermined object.

It is another object of the present invention to provide a method for the manufacture of a patterned composite membrane.

It is another object of the present invention to provide a method for the manufacture of a patterned composite membrane (and the like), the method being well-suited to industrial application involving comparatively large commercial volumes.

Other objects of the present invention will become apparent from the following detailed description taken in conjunction with the accompanying drawings.

### BRIEF DESCRIPTION OF THE DRAWINGS

Each of FIGS. 1 to 5 provide schematic representational illustrations. The relative locations, shapes, and sizes of objects have been exaggerated to facilitate discussion and presentation herein.

FIG. 1 is a schematic top view of a patterned composite membrane 10 according to an embodiment of the present invention.

FIG. 2 is a schematic side view of the patterned composite membrane 10 of FIG. 1, as seen along cross-section A-A therein.

FIG. 3 is a schematic side view of a mask 20 overlaid onto a substantially planar support 12 according to a method embodiment of the present invention.

FIG. 4 is a schematic side view of masks 20a, 20b, 20c, and 20d being sequentially overlaid onto a substantially planar support 12 according to another method embodiment of the present invention.

FIG. 5 schematically illustrates examples of varying arrangements of binding sites 14 provided in embodiments of the patterned composite membrane according to the present invention.

### DETAILED DESCRIPTION

The present invention provides in general a patterned composite membrane 10 that can be employed usefully in several and diverse analytical procedures, such as, but not limited to, the analytical procedures involved in proteomic and genomic biopolymer characterization. Fundamentally, the patterned composite membrane is used to selectively or preferentially capture, bind, isolate, remove, or otherwise withdraw from a fluid phase (i.e., an aqueous or gaseous phase) a chemically or mechanically separable component thereof as a result of interaction between said component and the patterned composite membrane 10. The targeted component is withdrawn into discrete regions, the pattern (or arrangement) and chemistry of which is predefined according to one's analytical objectives.

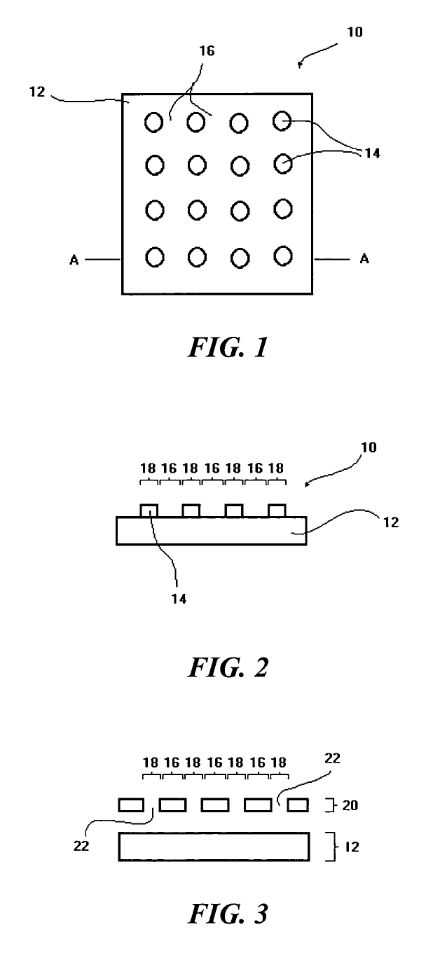

As illustrated in FIGS. 1 and 2, the patterned composite membrane 10 comprises a substantially planar support 12 onto which is provided porous material 14. In one embodiment, the porous material is a membrane-type material having porosity and microstructure capable of selectively admitting and retaining an object of predetermined size (e.g., particulate pollutants, bacteria, viruses, plant cells, animal cells, cell organelles, etc.). In a related embodiment, the porous material 14 comprises a plurality of particles dispersed in a polymeric binder and configured to preferentially bind a predetermined biopolymer (e.g., oligonucleotides, nucleic acids, polypeptides, etc.).

The porous material 14 is arranged on the substantially planar support 12 in a manner that defines a plurality of discrete regions 14, which—depending again on one's analytical objectives—can be configured to function as, for example, protein binding zones, immunochemical probes, hybridization reaction sites, or simply, discrete porous deposits capable of the aforementioned selective admission and retention of objects of predetermined size. Although the present invention is not confined in respect of whether each of its discrete regions 14 will have similar or different compositions or configurations, in all embodiments of the present invention, each discrete region 14 are fundamentally configured to chemically and/or mechanically differentiate between certain pre-defined target and non-target species.

The porous material useful in the present invention are those capable of being deposited—preferably, by the spray-cast methodology described further below—onto said substantially planar support 12 with an adhesivity and cohesivity sufficient to provide a patterned composite membrane 10 capable of undergoing a predetermined analytical procedure without substantial incidence of fracturing, erosion, fissuring, and/or other adhesive and cohesive failures. The porous material should also yield discrete regions 14 having rapid adsorption kinetics, a capacity and selectivity commensurate with one's predetermined analytical objectives, and—for certain applications—should allow for comparatively easy elution of bound analyte with an appropriate desorption agent.

Typically, the discrete regions 14 of porous material—particularly, when “spray-casted”—will not lay flush with the surface of the underlying support 12. Rather, the discrete regions 14 will have a certain thickness and bulk, analogous to raised relief structures, over the surface of the support 12. Such physical dimensionality increases the ratio of the surface area of the discrete regions to the surface area of the underlying support 18, thus advantageously increasing the immediate contact area available for binding/capture interactions. The physical dimensionality also increases the ratio of the volume of the discrete regions 14 to the surface area of the underlying support 18, thus advantageously increasing the region 14's binding/capture capacity, which itself can lead to the acquisition of stronger “signals” in post-sampling analysis.

Examples of useful porous materials include, but are not limited to, a fluoropolymer, a polyamide, a polyethersulfone, an acrylic, a polyester, or a cellulose ester. Preferably, the porous medium includes poly(vinylidene difluoride), polytetrafluoroethylene or a nylon, such as nylon-46, nylon-6, nylon-66 or nylon-610. For example, microporous filter media can be prepared using polyamides following the procedure of U.S. Pat. No. 4,340,479, using poly(vinylidene difluoride) following the procedure of U.S. Pat. Nos. 4,341,615 and 4,774,132, using polytetrafluoroethylene following the procedure of U.S. Pat. Nos. 3,953,566 and 4,096,227, using a polyethersulfone following the procedure of U.S. Pat. No. 5,480,554.

The currently desired porous material are these currently employed in the field of membranology. Such porous membrane material have been made by a variety of means including: (i) introducing a solution of a resin in a relatively good solvent into a solution which is a relatively poor solvent for the resin, e.g., as described in U.S. Pat. No. 4,340,479, (ii) by preparing a solution of a resin in a mixture of two solvents, one of which is a better solvent with a relatively higher vapor pressure compared with the second solvent, and allowing the solvents to evaporate, thereby forming a porous film, or (iii) as in the case of so-called “Teflon” membranes, by precipitating a suspension of finely particulate polytetrafluoroethylene (PTFE). It is believed that skilled membranologists, in view of the present disclosure, will know how to advantageously incorporate such membrane preparation techniques toward configuration of embodiments of the present invention.

A suitable membrane composition comprises about 80% w/w silica and 20% w/w polysulfone binder, and is produced by Millipore Corporation (Bedford, Mass.).

Functional composite structures comprising other micron-size (e.g., 1-30 microns) resin particles derivatized with other functional groups are also beneficial, including styrenedivinyl-benzene-based media (unmodified or derivatized with, for example, sulphonic acids, quarternary amines, etc.); silica-based media (unmodified or derivatized with C₂, C₄, C₆, C₈, or C₁₈, or ion exchange functionalities), to accommodate a variety of applications for peptides, proteins, nucleic acids, and other organic compounds. Those skilled in the art will recognize that other matrices with alternative selectivities (e.g., hydrophobic interaction, affinity, etc.) can also be used, especially for classes of molecules other than peptides.

The term “particles” as used herein is intended to encompass particles having regular (e.g., spherical) or irregular shapes, as well as shards, fibers and powders, including metal powders, plastic powders (e.g., powdered polystyrene), normal phase silica, fumed silica, and activated carbon. For example, the addition of fumed silica into a polysulfone polymer results in increased active surface area and is suitable for various applications. Polysulfone sold under the name UDEL P3500 and P1700 by Amoco is particularly preferred in view of the extent of the adherence of the resulting composite structure to the support 12. Other suitable polymer binders include polyethersulfone, cellulose acetate, cellulose acetate butyrate, acrylonitrile polyvinyl chloride copolymer (sold commercially under the name “DYNEL”), polyvinylidene fluoride (PVDF, sold commercially under the name “KYNAR”), polystyrene and polystyrene/acrylonitrile copolymer, etc.

Adhesion to the substantially planar support 12 can be enhanced or by an analogous effect achieved with these composite structures by means known to those skilled in the art, including etching of the substantially planar support 12, such as with plasma treatment or chemical oxidation. An intermediate adhesion layer (not shown) between the discrete regions 14 and the substantially planar support 12 can also be employed.

If a “spray-cast” particle-containing porous material is desired, consideration is advised on the influence of total particle concentration on casting solution viscosity and the influence of that viscosity on the conduct of spray-casting. In practice, it has been found that, depending on particle type, up to about 30% (w/w) of particles can be added to a typical polymeric matrix-forming solution without resulting in a viscosity unsuitable or otherwise undesirable for spray-casting. Greater particle loadings may be achieved using higher temperature. Suitable particle sizes include particles in the range of from about 100 nanometers to about 100 microns in average diameter.

In respect of the scope of the present invention, there is no general limitation as to whether the composition of the porous material 14 at each discrete region 14 is similar or different. Similarity or difference, and the extent thereof, will depend on the particular application to which the invention is drawn, and thus ultimately to the nature of the information which one wishes to obtain. In general, however, the less varied the information sought, the more similar the composition and/or configuration of the binding sites; the more varied the information sought, the greater the difference.

While one skilled in the art will be able to contemplate others, an example where each of the discrete regions 14 will have identical compositions is where the pattern of reactive sites is arranged to form a pictorial or textual image. Properly configured, the collective reaction (or lack thereof) of the reactive sites to a sample can essentially provide “On” and “Off” states that determine whether the pictorial or textual image is displayed or not. Such scheme has potential application, for example, in analytical protocols where the principal information sought is the presence or absence of a single or particularly restricted range or family of biopolymers, or contaminants, or pollutants, etc., such as pregnancy detectors, certain water analyses, and carbon monoxide detectors. The use of a resolvable image in this manner provides advantage by facilitating visual analysis of the patterned composite membrane 10, essentially reducing the level of requisite education and/or skills needed for interpretation and comprehension of sampled data.

For information-intense genomic and proteomic applications, the composition of the porous material should be varied and different at each discrete region 14 to effect a different biopolymeric specificity therein, and such that the resultant patterned composite membrane 10 can be used to extract distinct information at each discrete region 14. In embodiments wherein the porous material comprises particles dispersed in a binder, one means by which differentiation can be effected is by changing the composition of the particles at each discrete region 14. For example, as mentioned above, C18 particles can be used as the biopolymerically sorptive (or alternatively, “affinity-modified”) particles that are dispersed in the polymeric matrix material. And, C18 and like particles can be modified by conventional processes known by those skilled in the art—for example, the grafting of ligands on the particles for protein detection protocols.

As will be appreciated by those skilled in the art, the arrangement of the porous material into discrete regions 14 on the substantially planar support 12 is subject to variation. The patterns formed thereby can include both image-forming patterns (e.g., text, line-art graphics, icons, and symbology) and non-image-forming patterns (e.g., 2-dimensional and 3-dimensional planar dot arrays, grids, stripes, and concentric circles). The selection of the pattern will depend on the particular application sought for the patterned composite membrane 10, but in general, the image-forming patterns are well-suited for comparatively low information applications involving visual detection, with the non-image-forming patterns better suited for comparatively higher-information applications involving more sophisticated visual and/or machine-assisted detection and analysis.

While much latitude exists for the selection of a pattern for the discrete regions 14, in respect of application to biopolymer characterization protocols, the preferred arrangement is an array, in part because the regularity of said pattern facilitates easier visual and machine-assisted analysis, as well as present a more regular and ordered format for detailed biotechnical information. Regardless, array patterns are in themselves subject to variation. For example, in a so-called two-dimensional planar array, the individual discrete reactive sites are arranged in a rectangular grid pattern, such that they form rows and columns. When a more densely packed arrangement is desired, arranging the binding sites according to a hexagonal grid pattern (i.e., a three-dimensional planar array) will result in a plurality of rows and columns in which the rows and columns are not perpendicular, and accordingly, more space efficient arrangement.

Regardless of the type of array selected, one skilled in the art will appreciate that the particular shape of the discrete sites—when not contiguous—is generally unimportant. Such sites may be shaped as dots, rectangles, squares, hexagons, etc. Nonetheless, it should be apparent that facility in analysis is promoted in an array (or other) configuration by use of substantially similar shaped and sized binding sites.

In respect of microarray applications of the present invention, other factors potentially impinging upon pattern design can be considered. For example, it will be appreciated that as the spot density increases, spot size decreases, translating to a smaller number of recognition elements per spot. The sensitivity limit at spots of decreasing dimensions may become limited because of the dependence of DNA binding on the concentration of the immobilized probe. Also, if probe molecules are too densely packed on the microarray surface, hybridization of the biopolymeric target can be inhibited by steric interference. The upper limit for detection is proportional to the number of potential binding sites in the spot: the more binding sites, the larger the number of targets that can be captured. Those skilled in the art should be able to design an appropriate pattern based upon these and other considerations.

For so-called microarray applications, a particularly preferred pattern is the two-dimensional array. In this regard, two varieties have been considered: a contiguous array and a non-contiguous array of spots.

In the first variety, the porous material 14 is arranged on the substantially planar support 12 in a two-dimensional array of non-contiguous dots. Again, the dots themselves can be of any size and any shape, for example, round, square, rectangular, etc. Regardless, the non-contiguous spots are surrounded by areas 16 of the substantially planar support 12 that remain uncovered by the porous material 14. See e.g., FIG. 2. This first variety is particularly suitable in applications where ease of detection is more important than information density: cf., an array of non-contiguous spots are more likely to be more easily visually-detectable than an array of contiguous spots.

In the second variety, the porous material 14 is arranged on the substantially planar support to define a plurality of contiguous, but nonetheless, discrete reactive sites 14a, 14b, and 14c. In this regard, all relevant extents of the substantially planar support 12 are covered with a two-dimensional array of porous material 14. Each reactive sites is detectably differentiated from neighboring sites by composition and biopolymeric reactivity. The second variety is particularly suitable in applications where information density is more important that ease of detection: cf., a greater number of sites can be placed in a given unit area if they are contiguous.

Examples of a few of the patterns that can be employed according to certain embodiments of the present invention are illustrated in FIG. 5. In particular, FIG. 5(a) illustrates schematically a striped pattern of porous material 12 deposited onto substantially planar support 12. FIG. 5(b) illustrates schematically the two-dimensional array of non-contiguous spots 14 deposited onto substantially planar support 12. And, FIG. 5(c) illustrates schematically a two-dimensional array of contiguous spots 14a, 14b, 14c, etc., deposited onto, and covering in its entirety, substantially planar support 12.

Typically, once deposited onto substantially planar support 12, the porous material 12 cannot, without machine assistance, be visually detected, and the patterned membrane structure will appear upon casual inspection to be a uniform undifferentiated sheet or panel of media. However, when brought into contact with an appropriate target-containing sample for analysis at the appropriate conditions and for a sufficient time, interaction between the target and the porous material are effected. The type of interaction will depend naturally on the application design. For example, in a possible genomic application, a hybridization reaction can occur between a strand of polynucleic acid in solution and a complementary strand of polynucleic acid incorporated into the porous material of a binding site 14. Likewise, in a possible proteomic application, an immunochemical reaction can occur between an antigen in solution and an antibody therefor incorporated into the porous material of a binding site 14.

For size-based physical separations—i.e., where the target is an object of predetermined size—the interaction between the porous material and the targeted object can be purely mechanical in character. For example, the porous material 14 can be configured to have a microstructure comprising a random matrix of essentially chemically-inert fibers bonded to form a complex maze (or network) of flow channels. An object carried in a fluid phase, having a physical dimension below the nominal pore size attributable to deposited porous material 14, is selectively admitted into microstructure of the porous material 14, where it becomes lodged or otherwise entrapped, and thus retained for subsequent analysis. Since it is currently difficult to control nominal pore size in fiber matrices, analytical applications of patterned composite membranes 10 having such microstructure (and structural and/or functional equivalents thereof) may yield comparatively rough target discrimination. Regardless, as known to those skilled in the art, target discrimination can be improved by careful and controlled target sample preparation.

In view of the broad range of possible applications, the methods by which detection of the biopolymeric reaction can be accomplished are several. These may involve, for example, visual detection, staining, fluorescence, microscopic analysis, radioactive labeling, etc.

In respect of the aforementioned patterned composite membrane 10 employing a striped pattern of reaction sites, detection can be accomplished by the use of a scanning fluorimeter, the use of which is disclosed for example in U.S. Pat. No. 4,076,420, issued to DeMaeyer et al. on Feb. 28, 1928; U.S. Pat. No. 4,942,303, issued to Kolber et al. on Jul. 17, 1990; and U.S. Pat. No. 5,894,347, issued to MacDonald on Apr. 13, 1999. In detection, the scan direction of the fluorimeter can be aligned with the pattern of stripes of the patterned composite membrane 10 such that scan line will correspond with the stripes, thereby allowing a smooth, sweeping machine-assisted analysis of the array.

In contrast to machine-assisted post-treatment analysis of the patterned composite membrane 10, certain applications may require only visual detection. For example, in applications where the target object is, but not necessarily, a biopolymer, there are—particularly in the thin layer chromatographic arts—several known methods for staining such molecules, thus rendering their presence known by visual inspection. It is further envisaged that the additional chemistries can be incorporated into the discrete binding sites such that no further staining would be required for visual analysis, for example, a chemistry that would produce a distinct chromophore upon contact of the binding site with its target. Such chemistry can be time and concentration sensitive such that greater or less chromophore is produced under corresponding conditions, thus providing further observable information for analysis. It is envisaged, that in such applications, the pattern selected for the reactive sites will facilitate further analysis of the treated patterned composite membrane 10.

Additional potentially-applicable analytical processes are derivable from (or can be derived from) methodologies and techniques currently employed in the so-called “western blotting”, “northern blotting”, and “southern blotting” protocols. Western blotting is a method for detecting or transferring proteins and is generally described in Towbin et al., Proc. Natl. Acad. Sci. USA, 76, 4350-4354 (1979). Northern blotting is a method for detecting or transferring RNA's and is generally described in Thomas, Proc. Natl. Acad. Sci. USA, 77, 5201-5205 (1980). Southern blotting is a method for detecting or transferring DNA's and is generally described in Southern, J. Mol. Biol., 98, 503-517 (1975). These detection or transfer methods, as well as numerous variations thereon and other detection or transfer procedures utilizing membranes, particularly hydrophobic membranes such as polyvinylidene fluoride membranes, are well-known in the art.

In selecting materials for the substantial planar support 12, consideration is to be given to the exclusion of materials that may disrupt, interfere with, or otherwise effect undesirably the occasion and/or subsequent detection of the preferential reaction between the porous material 14 and the predetermined target under investigation. For example, if the porous material 14 and the material employed for the substantially planar support 12 equally attract and bind the target under investigation, the diagnostic value of the patterned composite membrane 10 is diminished due to increasing background noise and decreasing signal-to-noise ratio for the target material. While this may be an extreme case, those skilled in the art will appreciate that most organic materials will to some extent bind biopolymers, so it is difficult to identify for use support material that is absolutely inert to the biopolymer under investigation. While a support 12 that is absolutely inert is preferred, in practice, it may be sufficient if the support 12 is only comparatively inert relative to the affinity to the target of the porous material 14 sufficient to allow reasonably accurate detection of a captured target.

Apart from being comparatively inert to the target, there is no particular limitation to the substantially planar support 12 other than that the polymer material should adhere to it. The support 12 can be porous or non-porous.

Examples of materials for the substantially planar substrate 12 include, but are not limited to, non-woven polyolefin fabrics, microporous UPE membranes, polypropylene, polyvinyly chloride, polycarbonate, polytetrafluoroethylene, polyvinylidiene fluoride, mixed cellulose esters, polyether sulfone, nylon, high-density polyethylene, polypropylene, polystyrene, modified acrylics, polyethylene terephthalate, glass, and stainless steel.

Although many materials having desirable physical properties may suffer from poor adhesivity and reactive incompatibility, the treatment of said materials, for example, by application thereon of adhesion promoting, biologically inert coatings, can cure such deficiencies. Hence, in construing the scope of the present invention, it should not be inferred that the substantially planar support is—in its composition, construction, and/or material properties—a homogenous and/or unitary structure. To the contrary, for certain applications, advantage may be employed, for example, by employing a substantially planar substrate comprising a plurality of lamina, each having a number of other functions.

There is no particular limitation to the size and the shape of he substantially planar support 12 in practice of the present invention. However, if the substantially planar support 12 comprises porous membrane-type material, one should consider the several current devices and housings that incorporate and/or use such membranous media. Shaping and sizing said membrane support for installation in or compatibility with such devices and housings may be advantageous. For example, under current so-called microarray-based bioanalytical procedures—i.e., the aforementioned deposition, reaction, and analysis of samples on glass slides—the diffusion rate of a sample to and through a pre-sensitized biochemical probe is typically slow, and thus a rate limiting factor. The present invention offers an alternative. Properly shaped and sized, the patterned composite membrane 12 can be incorporated into a housing that is compatible with existing vacuum filtration apparatus such that sampling can be conducted quickly and efficiently under a vacuum. The diffusion rate should be comparatively improved.

It should be appreciated that, in certain embodiments of the present invention, the region 18 of substantially planar support 12 onto which porous material is deposited is not synonymous with the physical boundaries of the discrete binding sites 14. Particularly in the case of biopolymeric sample analysis, it may be desirable to first deposit large areas (or area) of porous materials onto the substantially planar support 12, then subsequently differentiating discrete reactive sites within some or all of those larger area, for example, by a post-deposition treatment that modifies the biopolymeric reactive functionality of the membrane material therein. In such embodiments, the binding sites 12, though discrete, will likely be contiguous. Cf., FIG. 5(c), discussed supra.

As mentioned, each binding site is configured to preferentially select (chemically or mechanically) a predetermined biopolymer. The selection should be “preferential” in the sense that reaction with the targeted biopolymer will occur to the substantial exclusion of reaction with non-targeted species (e.g., other non-targeted biopolymers, salts, etc.) that may be contained in a sample. Chemical interaction would involve, for example, hybridization, immunochemical binding, adsorption, and other organic reactions involving covalent, ionic, and/or hydrogen bonding. Certain of these processes, may in respect of certain targeted biopolymers be comparatively slow, and thus preferential selection can be improved by external influences, such a by shaking, bubbling, and other means of generating convective fluids.

In the embodiments of the present invention, in which the targeted unit is not a specific predetermined biopolymer, but rather, for example, a particle, cell, or cell component, preferentially selection thereof, can also include, for example, sized-based mechanical selection. For example, the deposited porous material may in itself be inert, but has a microstructure of predefined porosity, or contain beads of predefined porosity, that function to selectively entrap particles of certain dimension. In essence, the porous material has a porosity and microstructure capable of preferentially admitting and holding an object of predetermined size.

In a desirable embodiment of the inventive patterned composite membrane 10, wherein the substantially planar support 12 comprises a porous polymeric composition that is substantially unreactive with the predetermined target, both the substantially planar support 12 and the discrete binding sites 14 are configured to be substantially hydrophilic, regardless of the similarity or difference of their specific composition. The overall hydrophilicity of its components improves the so-called “wetability” of the resultant patterned composite membrane 10, as well as reduce its requisite “liquid initiation/penetration pressure” threshold. These improvements are particularly advantageous in applications involving an analysis of a liquid sample and the processing thereof in a vacuum filtration apparatus.

It is contemplated that a user of a patterned composite membrane 10 may wish only to use a single unit to obtain a single set of information for a single application, and in which case, a single patterned composite membrane 10 may be custom assembled by said practitioner. However, the generally inexpensive configuration of the array 10 is well-suited for and invites applications involving several uses of several units, for example, to confirm analytical results or to characterize a wide range of biopolymeric samples. In this regard, need exists for a method for the manufacture of the patterned composite membrane 10 that is uncomplicated, can be operated at a comparatively fast rate, and can produce at high yields at a consistent quality. A “mask-based stenciling methodology”—i.e., a method in which a mask is used to form a pattern of membrane precursor material onto a substantially planar support—meets this need.

The starting materials used in the mask-based methodology are (a) the aforementioned substantially planar support 12 and (b) a membrane precursor solution capable of being processed to form the aforementioned porous material capable of selectively admitting and retaining an object of predetermined size.

The materials useful for the substantially planar support 12 are the same as mentioned above.

Likewise, the useful membrane precursor solutions are those that can yield the aforementioned porous material. However, it will be appreciated that the methodology can be practiced to manufacture patterned arrays other than the patterned composite membrane 10, i.e., patterned arrays that do not necessarily incorporate sorptive particles and/or porous material. Hence, other curable polymeric solutions can be employed with the same broad advantages otherwise accomplished in the methodology. Thus, although polysulfone, polystyrene, and cellulose acetate (with and without particles) are currently preferred, there is no particular limitation to the polymer lacquers that can be employed in the practice of the inventive methodology.

Provided with a suitable substantially planar support 12 and a membrane precursor solution, the method proceeds by superposing a mask or stencil (hereinafter, mask 20) over the substantially planar support 12 (as shown in FIG. 3), and bringing them into intimate contact.

The mask 20 will generally comprise a sheet material with at least one opening, hole, aperture, or bore therethrough (collectively hereinafter, “opening 22”). The opening 22 has dimensions sufficient for the facilitated or un-facilitated passage therethrough of the membrane precursor solution. In respect of its functionality as an imaging tool, it will be appreciated that mask 20 is essentially “negative-working”. In other words, in those areas 18 of the support onto which deposition of material is desired, an opening 22 in the mask 20 is provided; whereas in those areas 16 where deposition of material is not desired, no opening is provided.

In a currently preferred mode of practice, the mask 20 has a thickness less than about 0.1″ (0.254 cm.) and is capable of laying substantially flat on either a flat plane (e.g., such as found on a flat-bed type stenciling apparatus) or on a cylindrical plane (e.g., such as found on a rotary drum type stenciling apparatus). In respect of certain currently-preferred spot patterns, deposition of a multiplicity of 0.015″ (0.0381 cm.) diameter membrane spots is favored, with the centers thereof separated by approximately 0.030″ (0.0762 cm.).

Achieving intimate contact between the mask 20 and the substantially planar support 12 is important to obtaining a sharp, well-resolved pattern. Lose contact can lead to solution dispersion on the surface of the substantially planar support 12, particularly if the membrane precursor solution has comparatively low viscosity. Means of attaching the mask could be mechanical (e.g., clamps) or chemical (e.g., adhesive). Details of various attaching means are disclosed, for example, in U.S. Pat. No. 4,223,602, issued to M. Mitter on Sep. 23, 1980; U.S. Pat. No. 3,941,054, issued to E. M. Springer on Mar. 2, 1976; U.S. Pat. No. 3,980,017, issued to J. A. Black on Sep. 14, 1976; and U.S. Pat. No. 4,060,030, issued to F. J. Noschese on Nov. 29, 1977.

With intimate contact between the mask 20 and the substantially planar support 12 accomplished, the membrane precursor solution is then deposited onto the substantially planar support 12 through said opening(s) 22 of said overlaying mask 20. The most preferred method of accomplishing deposition is spraying.

Methods for spraying polymeric compositions are several and well-known in the coating arts, many of such application having applicability to the present invention. In general, however, spraying means will generally comprise a fluid dispersion nozzle having an appropriately shaped and sized aperture through which the polymeric solution is propelled at a velocity and pressure, in combination with or under the influence of an inert propellant, sufficient to effect dispersal of an expanding forward projection of said polymer solution. For certain polymer solutions, additives may be needed to modify the viscosity and/or other rheological properties of the solution to enable the spraying thereof.

Other methods of deposition include, for example, brushing, slot coating, knife coating, curtain coating, sputtering, and the like. In the formation of reactive sites incorporating sensitive biopolymerically-active constituents, consideration should be given to the selection of a suitable deposition method that will not destroy, disrupt, or otherwise interrupt the bioreactivity of said constituents.

In the circumstance, for example, where the membrane precursor solution has comparatively high viscosity and the dimensions for the mask opening(s) 22 are comparatively minute, passage of the membrane precursor solution through the opening(s) 22 may be difficult, if not facilitated. The use of a vacuum can facilitate solution passage, as can the use of mechanical means of exerting pressure onto the precursor solution (e.g., use of a squeegee or roller).

Following deposition, the mask 20 is removed from the substantially planar support 12 at a time and manner such that the deposited membrane precursor solution remains on the support. As will be appreciated by the skilled practitioner, the viscosity (as well as other rheological and/or material properties) of certain membrane precursor solutions can change as a function of time and environmental conditions. If the deposited precursor solution becomes, for example, too viscous or too hard, it may become difficult to remove the mask 20 without also removing portions of the porous material 14, or tearing surrounding areas of the substantially planar support 12, or otherwise damaging or yielding unfit for use the resultant patterned composite membrane 10. Even slight damage may under certain conditions diminish the diagnostic value of the resultant patterned composite membrane 10. Thus, when using high viscosity solution, measures should be taken to control such problems, for example, by reducing the viscosity of the precursor solution, or by removing the mask 20 prior to the complete setting of the solution, or by incorporating additives into the precursor solution to modify certain of its physical properties (e.g., its cohesivity, fracturability, etc.).

In preferred practice, the porous material deposition 14 remaining on the substantially planar support 12 should correspond substantially to the shape of said opening(s) 22 of said mask 20.

Before or after the removal of the mask 20, the membrane precursor solution is processed to form said porous material 14. A typical process involves contacting the deposited membrane precursor solution with a liquid or vapor in which the polymer contained therein is insoluble, preferably water, so that the polymer precipitates in the housing. This can be accomplished by immersing the yet unfinished patterned membrane array in the liquid, and/or otherwise applying the liquid onto the deposited membrane precursor solution. Through the exchange of water for the solvent, the structure precipitates. Those skilled in the art will appreciate that the solvent used to prepare the casting solution and the non-solvent can contain a variety of additives.

The quenching bath can be aqueous, non-aqueous, or a mixture at approximately 5 to 55 degrees centigrade. Depending on the desired permeability, the membrane can be precipitated selectively from either side by floating the substrate or be immersed in its entirety.

In accordance with the present invention, the structures of the present invention can be formed by a polymer phase inversion process, air casting (evaporation), and thermal inversion.

In the polymer phase inversion process, the solvent for the polymer must be miscible with the quench or inversion phase. For example, N-methyl-pyrolidone is a solvent for polysulfones, polyethersulfones, and polystyrene. In the latter case, polystyrene pellets can be dissolved in N-methyl-prolidone and spray casted. The resulting structure shows good adhesion to many desirable supports, and has adsorption characteristics similar to polysulfone. Dimethylsulfoxide (DMSO), dimethylformamide, butyrolactone, and sulfalane are also suitable solvents. N,N-dimethylacetamide (DMAC) is a suitable solvent for PVDF. Water is a preferred precipitant.

In the air casting process, a volatile solvent for the polymeric binder is used. For example, in the case of cellulose acetate, acetone is a suitable volatile solvent. The solvent can be simply evaporated off or exchanged with water vapor in a humidity chamber. The latter yields more porous structures.

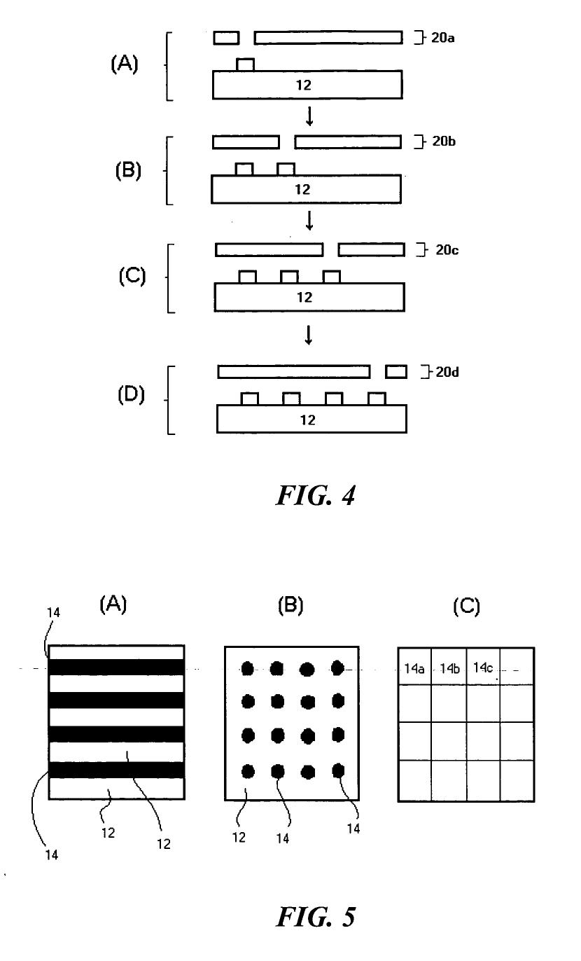

In the practice of the inventive methodology, it will be appreciated that the material deposition by the use of a mask 20 to provide a finished pattern can be accomplished either sequentially or in an overall, so-called “blanket-wise” manner. Blanket-wise deposition is illustrated in FIG. 3. Sequential deposition is illustrated in FIG. 4.

In blanket-wise deposition, the entire pattern of binding sites is deposited onto a substantially planar support 12 in a single step. All the openings 22 needed for the desired pattern are provided on the mask 20. Thus, applying material through such mask onto the support can yield in a single step the final pattern. This is advantageous where speed and simplicity of deposition is desired. However, it will be appreciated that since only a single deposition step is involved, only one type of polymeric membrane precursor material is deposited, and accordingly, the composition of the deposited regions will be virtually identical. Thus, if differentiation among deposited regions is desired, such must be accomplished by other post-deposition methodologies. In respect of applicability to industrial manufacture, the blanket-wise deposition methodology is particularly well-suited for, but not necessarily limited to, a flat-bed type stenciling operation utilizing so-called “step-and-repeat” manufacturing line procedures.

Where a more complex pattern is desired, such as those wherein there is much compositional differentiation among the binding sites, one may wish to repeat the steps of the inventive methodology in sequential stages to gradually build up the final desired pattern. In particular, by repeating the process using the same substantially planar support, one can build sequentially and or by layers a complex pattern of binding sites with varying material constituency through the sequential use of different masks and different curable polymeric solutions at each reiteration of the process.

FIG. 4 illustrates a sequential process in which the substantially planar support 12 onto which material to be deposited is intermittently advanced through a series of deposition stations (a), (b), (c), and (d). After every advancing step, a stencil (20a, 20b, 20c, 20d) is lowered onto the substantially planar support 12, the membrane precursor solution to be applied by screen printing is admitted onto the upper surface of the mask 20, and a squeegee then squeezes the medium through the stencil perforations and onto the substantially planar support 12. Depending in part on the properties of the curable polymeric solution, a curing station can be position between each deposition stations to cure the just-deposited polymeric solution to prevent it from being smeared, smudge, blotched, or otherwise disturbed by subsequent deposition procedures.

In respect of applicability to industrial manufacture, the step-wise deposition methodology is particularly well-suited for, but not necessarily limited to, a rotary type stenciling operation utilizing so-called “continuous” manufacturing line procedures. Those skilled in the art will appreciate that the use of rotary drum-based deposition admits of manufacture onto a continuous web of support material. Such continuous web-based manufacture is advantageous where volume and yield of product are important concerns. Examples of the use of stencils on a rotary drum are discloses, for example, in U.S. Pat. No. 3,948,169, issued to J. R. Cole on Apr. 6, 1976; and U.S. Pat. No. 4,107,003, issued to L. Anselrode on Aug. 15, 1978.

Regardless of whether deposition is preformed in a blanket-wise or step-wise manner, the most preferred manner of practicing the inventive methodology is the aforementioned spray casting technique. By spraying the material onto the substantially planar support 12 through the mask 20, several advantages are realized which would not be attainable using a more direct physical deposition of the material. In particular, because spraying does not require physical contact with the mask, the potential for unintentionally shifting, raising, tearing, creasing, bending and/or otherwise displacing or damaging the mask 20 during the deposition step—all of which can result in unwanted and/or accidental deposition anomalies—is reduced. Spraying also can be effected with good coverage, speed, uniformity, and control.

### EXAMPLE

A 5″×5″ piece of Freudenberg 2439 polyolefin fabric substrate (i.e., a substantially planar support) was taped by its corners to a 12″×12″×0.25″ glass plate. To this, a stainless steel mask (2″×3.5″×0.004″) containing several patterns was firmly taped around the edges to the center of the substrate. A “C18 lacquer” (i.e., a membrane precursor solution) comprising 9% UDEL P3500 polysulfone/91% n-methyl-pyrrolidone with 29% (w/w) c18-200-15sp spherical particles was then loaded into the reservoir of an airbrush. The airbrush was adjusted to deliver a fine spray of lacquer using 50 psi (8.66 kg/cm²) of air pressure. The glass plate was laid flat on a sturdy horizontal surface and, while gently pushing down on the metal stencil, lacquer is carefully sprayed onto the pattern in moderation. Upon completion of spraying, the substrate was gently removed from the glass plate with the mask still attached and floated back side down on the surface of a water bath at room temperature for about 5 minutes followed by total immersion for about a half hour. After this period, the substrate was removed, the mask peeled off, and the resultant patterned composite membrane allowed to air dry.

1. A patterned composite membrane, useful for proteomic and genomic biopolymer characterization, comprising: a substantially planar support onto which is provided a plurality of discrete binding sites arranged in a predetermined pattern, each binding site being composed of a porous material, the porous material comprising a plurality of particles dispersed in a polymeric matrix, said particles configured to preferentially bind a predetermined biopolymeric species.

2. The patterned composite membrane of claim 1, wherein the substantially planar support comprises a porous polymeric composition that is substantially unreactive with said predetermined biopolymer, and wherein both the substantially planar support and the discrete binding sites are substantially hydrophilic.

3. The patterned composite membrane of claim 1, wherein the porous material is arranged on the substantially planar support in a two-dimensional or three-dimensional planar array of non-contiguous spots, the non-contiguous spots being surrounded by areas of the substantially planar support uncovered by the porous material.

4. The patterned composite membrane of claim 1, wherein the porous material is arranged on the substantially planar support to define a plurality of contiguous discrete binding sites, the discrete binding sites being detectably differentiated by composition and biopolymeric reactivity.

5. The patterned composite membrane of claim 1, wherein the porous material is arranged on the substantially planar support in a pattern of stripes.

6. The patterned composite membrane of claim 1, wherein said particles at each of said binding sites is configured to bind the same predetermined biopolymeric species.

7. A method for extracting a predetermined biopolymeric species from a solution comprising the steps of:
(a) providing a patterned composite membrane, the patterned composite membrane comprising a substantially planar support onto which is provided a plurality of discrete binding sites arranged in a predetermined pattern, each reactive site being composed of porous material, at least one of said binding sites being configured to preferentially bind said predetermined biopolymeric species;
(b) providing a solution containing said predetermined biopolymeric species; and
(c) treating said configured binding sites with said solution for a time and under conditions sufficient for said configured binding sites to preferentially bind said predetermined biopolymeric species.

8. A patterned composite membrane comprising a substantially planar support onto which is provided discrete non-contiguous deposits of porous material, each discrete deposit of porous material having a porosity and microstructure capable of selectively admitting and retaining an object of predetermined size.

9. The patterned composite membrane of claim 8, wherein said porous material at each discrete deposit comprises porous beads dispersed in a polymeric matrix.

10. The patterned composite membrane of claim 8, wherein said porosity and microstructure differ among said discrete deposits of said porous material.

11. The patterned composite membrane of claim 8, wherein the substantially planar support comprises a porous polymeric composition having a porosity and microstructure incapable of retaining said object of predetermined size, and wherein both the substantially planar support and the discrete reactive sites are substantially hydrophilic.

12. A method for extracting an object of predetermined size from a fluid phase comprising the steps of:
(a) providing a patterned composite membrane, the patterned composite membrane comprising a substantially planar support onto which is provided discrete non-contiguous deposits of porous material, at least one of said discrete deposits being configured to have a porosity and microstructure capable of selectively admitting and retaining said object of predetermined size;
(b) providing a fluid phase containing said object of predetermined size; and
(c) treating said configured discrete deposits with said fluid phase for a time and under conditions sufficient for said discrete deposits to selectively admit and retain said object of predetermined size.

13. A method for the manufacture of a patterned membrane array, the method comprising the steps of:
(a) providing a substantially planar support;
(b) providing a membrane precursor solution capable of being processed to form a porous material;
(c) overlaying a mask onto said substantially planar support, said mask comprising a sheet material with at least one opening therethrough, the opening having dimensions sufficient for the facilitated or unfacilitated passage of said curable polymeric solution therethrough;
(d) depositing said membrane precursor solution onto said substantially planar support through said opening of said overlaying mask;
(e) removing said mask from said substantially planar support so that the deposition of said membrane precursor solution remains on the support, said deposition corresponding substantially to the shape of said opening of said mask; and
(f) processing said membrane precursor solution to form said porous material.

14. The method of claim 13, wherein the step of depositing said membrane precursor solution is accomplished by spraying said solution through the opening of said overlaying mask.

15. The method of claim 13, wherein said mask comprises a plurality of openings, said opening being arranged according to a predetermined pattern, said predetermined pattern being a two- or three-dimensional planar array of non-contiguous areas, said non-contiguous areas having substantially similar shape and size.

16. The method of claim 13, wherein said membrane precursor solution comprises a polymeric-matrix forming material and sorptive or reactive particles, said particles being sorptive of or reactive with a predetermined biopolymer.

17. The method of claim 13, wherein said step of removing said mask from said substantially planar support is performed subsequent to said step of processing said membrane precursor solution.
